(19) United States
(12) Patent Application Publication
(10) Pub. No.: US 2004/0146960 A1
(43) Pub. Date: Jul. 29, 2004

(54) Methods for the identification of inhibitors of Trehalose-6-Phosphate Synthase as antibiotics

(76) Inventors: Blaise Darveaux, Hillsborough, NC (US); Sanjoy Mahanty, Chapel Hill, NC (US); Ryan Heiniger, Raleigh, NC (US); Amy Covington, Raleigh, NC (US); Huaqin Pan, Apex, NC (US); Rex Tarpey, Apex, NC (US); Jeffrey Shuster, Chapel Hill, NC (US); Matthew M. Tanzer, Durham, NC (US); Lisbeth Hamer, Durham, NC (US); Kiichi Adachi, Tokyo (JP); Todd DeZwaan, Apex, NC (US); Sze-Chung Lo, Shun Lee Estate (HK); Maria Victoria Montenegro-Chamorro, Morrisville, NC (US); Sheryl Frank, Durham, NC (US)

Correspondence Address: PARADIGM GENETICS, INC, 108 ALEXANDER DRIVE, P O BOX 14528, RTP, NC, 27709-4528, US

(21) Appl. No.: 10/703,292

(22) Filed: Nov. 7, 2003

Related U.S. Application Data
(60) Provisional application No. 60/424,868, filed on Nov. 8, 2002.

(51) Int. Cl. C12Q 1/48

(52) U.S. Cl. 435/15

(57) ABSTRACT

The present inventors have discovered that Trehalose-6-Phosphate Synthase is essential for normal fungal pathogenicity. Specifically, the inhibition of Trehalose-6-Phosphate Synthase gene expression in fungi results in reduced pathogenicity (i.e. smaller, non-viable lesions). Thus, Trehalose-6-Phosphate Synthase can be used as a target for the identification of antibiotics, preferably antifungals. Accordingly, the present invention provides methods for the identification of compounds that inhibit Trehalose-6-Phosphate Synthase expression or activity. The methods of the invention are useful for the identification of antibiotics, preferably antifungals.

### RELATED APPLICATIONS

This application claims the benefit of U.S. Provisional Application No. 60/424,868, filed Nov. 8, 2002. The foregoing patent application is incorporated in its entirety by reference.

### FIELD OF THE INVENTION

The invention relates generally to methods for the identification of antibiotics, preferably antifungals that affect the biosynthesis of trehalose.

### BACKGROUND OF THE INVENTION

Filamentous fungi are causal agents responsible for many serious pathogenic infections of plants and animals. Since fungi are eukaryotes, and thus more similar to their host organisms than, for example bacteria, the treatment of infections by fungi poses special risks and challenges not encountered with other types of infections. One such fungus is Magnaporthe grisea, the fungus that causes rice blast disease, a significant threat to food supplies worldwide. Other examples of plant pathogens of economic importance include the pathogens in the genera Agaricus, Alternaria, Anisogramma, Anthracoidea, Antrodia, Apiognomonia, Apiosporina, Armillaria, Ascochyta, Aspergillus, Bipolaris, Bjerkandera, Botryosphaeria, Botrytis, Ceratobasidium, Ceratocystis, Cercospora, Cercosporidium, Cerotelium, Cerrena, Chondrostereum, Chryphonectria, Chrysomyxa, Cladosporium, Claviceps, Cochliobolus, Coleosporium, Colletotrichium, Colletotrichum, Corticium, Corynespora, Cronartium, Cryphonectria, Cryptosphaeria, Cyathus, Cymadothea, Cytospora, Daedaleopsis, Diaporthe, Didymella, Diplocarpon, Diplodia, Discohainesia, Discula, Dothistroma, Drechslera, Echinodontium, Elsinoe, Endocronartium, Endothia, Entyloma, Epichloe, Erysiphe, Exobasidium, Exserohilum, Fomes, Fomitopsis, Fusarium, Gaeumannomyces, Ganoderma, Gibberella, Gloeocercospora, Gloeophyllum, Gloeoporus, Glomerella, Gnomoniella, Guignardia, Gymnosporangium, Helminthosporium, Herpotrichia, Heterobasidion, Hirschioporus, Hypodermella, Inonotus, Irpex, Kabatiella, Kabatina, Laetiporus, Laetisaria, Lasiodiplodia, Laxitextum, Leptographium, Leptosphaeria, Leptosphaerulina, Leucytospora, Linospora, Lophodermella, Lophodermium, Macrophomina, Magnaporthe, Marssonina, Melampsora, Melampsorella, Meria, Microdochium, Microsphaera, Monilinia, Monochaetia, Morchella, Mycosphaerella, Myrothecium, Nectria, Nigrospora, Ophiosphaerella, Ophiostoma, Penicillium, Perenniporia, Peridermium, Pestalotia, Phaeocryptopus, Phaeolus, Phakopsora, Phellinus, Phialophora, Phoma, Phomopsis, Phragmidium, Phyllachora, Phyllactinia, Phyllosticta, Phymatotrichopsis, Pleospora, Podosphaera, Pseudopeziza, Pseudoseptoria Puccinia, Pucciniastrum, Pyricularia, Rhabdocline, Rhizoctonia, Rhizopus, Rhizosphaera, Rhynchosporium, Rhytisma, Schizophyllum, Schizopora, Scirrhia, Sclerotinia, Sclerotium, Scytinostroma, Septoria, Setosphaera, Sirococcus, Spaerotheca, Sphaeropsis, Sphaerotheca, Sporisorium, Stagonospora, Stemphylium, Stenocarpella, Stereum, Taphrina, Thielaviopsis, Tilletia, Trametes, Tranzschelia, Trichoderma, Tubakia, Typhula, Uncinula, Urocystis, Uromyces, Ustilago, Valsa, Venturia, Verticillium, Xylaria, and others. Related organisms are classified in the oomycetes classification and include the genera Albugo, Aphanomyces, Bremia, Peronospora, Phytophthora, Plasmodiophora, Plasmopara, Pseudoperonospora, Pythium, Sclerophthora, and others. Oomycetes are also significant plant pathogens and are sometimes classified along with the true fungi. Human diseases that are caused by filamentous fungi include life-threatening lung and disseminated diseases, often a result of infections by Aspergillus fumigatus. Other fungal diseases in animals are caused by fungi in the genera Fusarium, Blastomyces, Microsporum, Trichophyton, Epidermophyton, Candida, Histoplamsa, Pneumocystis, Cryptococcus, other Aspergilli, and others. The control of fungal diseases in plants and animals is usually mediated by chemicals that inhibit the growth, proliferation, and/or pathogenicity of the fungal organisms. To date, there are less than twenty known modes-of-action for plant protection fungicides and human antifungal compounds.

A pathogenic organism has been defined as an organism that causes, or is capable of causing disease. Pathogenic organisms propagate on or in tissues and may obtain nutrients and other essential materials from their hosts. A substantial amount of work concerning filamentous fungal pathogens has been performed with the human pathogen, Aspergillus fumigatus. Shibuya et al., 27 Microb. Pathog. 123 (1999) have shown that the deletion of either of two suspected pathogenicity related genes encoding an alkaline protease or a hydrophobin (rodlet), respectively, did not reduce mortality of mice infected with these mutant strains. Smith et al., 62 Infect. Immun. 5247 (1994) showed similar results with alkaline protease and the ribotoxin restrictocin; Aspergillus fumigatus strains mutated for either of these genes were fully pathogenic to mice. Reichard et al., 35 J. Med. Vet. Mycol. 189 (1997) showed that deletion of the suspected pathogenicity gene encoding aspergillopepsin (PEP) in Aspergillus fumigatus had no effect on mortality in a guinea pig model system, whereas Aufauvre-Brown et al., 21 Fungal. Genet. Biol. 141 (1997) showed no effects of a chitin synthase mutation on pathogenicity.

However, not all experiments produced negative results. Ergosterol is an important membrane component found in fungal organisms. Pathogenic fungi lacking key enzymes in the ergosterol biochemical pathway might be expected to be non-pathogenic since neither the plant nor animal hosts contain this particular sterol. Many antifungal compounds that affect the ergosterol biochemical pathway have been previously described. (U.S. Pat. Nos. 4,920,109; 4,920,111; 4,920,112; 4,920,113; and 4,921,844; Hewitt, H. G. Fungicides in Crop Protection Cambridge, University Press (1998)). D'Enfert et al., 64 Infect. Immun. 4401 (1996) showed that an Aspergillus fumigatus strain mutated in an orotidine 5′-phosphate decarboxylase gene was entirely non-pathogenic in mice, and Brown et al. (Brown et al., 36 Mol. Microbiol. 1371 (2000)) observed a non-pathogenic result when genes involved in the synthesis of para-aminobenzoic acid were mutated. Some specific target genes have been described as having utility for the screening of inhibitors of plant pathogenic fungi. U.S. Pat. No. 6,074,830 to Bacot et al., describes the use of 3,4-dihydroxy-2-butanone 4-phosphate synthase, and U.S. Pat. No. 5,976,848 to Davis et al. describes the use of dihydroorotate dehydrogenase for potential screening purposes.

There are also a number of papers that report less clear results, showing neither full pathogenicity nor non-pathogenicity of mutants. Hensel et al. (Hensel, M. et al., 258 Mol. Gen. Genet. 553 (1998)) showed only moderate effects of the deletion of the areA transcriptional activator on the pathogenicity of Aspergillus fumigatus.

Therefore, it is not currently possible to determine which specific growth materials may be readily obtained by a pathogen from its host, and which materials may not. We have found that Magnaporthe grisea that cannot synthesize their own trehalose exhibit reduced pathogenicity (i.e. smaller, non-viable lesions) on their host organism.

Trehalose-6-phosphate synthase catalyzes the first step in the biosynthesis of the non-reducing disaccharide, trehalose. In both Saccharomyces cerevisiae and Aspergillus nidulans, increased sensitivity to stress and poor spore germination have been observed in strains lacking trehalose-6-phosphate synthase activity (De Virgilio et al., 219 Eur. J. Biochem. 179-186 (1994); Fillinger et al., 147 Microbiol. 1851-1862 (2001)). Disruption of trehalose-6-phosphate synthase in Candida albicans has been shown to reduced infectivity (Zaragoza et al. 180 J. Bacteriol. 3809-3815 (1998)). Published literature fails to demonstrate an antipathogenic effect of the knock-out, over-expression, antisense expression, or inhibition of the genes or gene products involved in trehalose biosynthesis in filamentous fungi. Thus, it has not been shown that the de novo biosynthesis of trehalose is essential for fungal pathogenicity toward plant hosts. Thus, it would be desirable to determine the utility of the enzymes involved in trehalose biosynthesis for evaluating antibiotic compounds, especially fungicides. If a fungal biochemical pathway or specific gene product in that pathway is shown to be required for fungal pathogenicity, various formats of in vitro and in vivo screening assays may be put in place to discover classes of chemical compounds that react with the validated target gene, gene product, or biochemical pathway, and are thus candidates for antifungal, biocide, and biostatic materials.

### SUMMARY OF THE INVENTION

The present inventors have discovered that in vivo disruption of the gene encoding Trehalose-6-Phosphate Synthase in Magnaporthe grisea prevents or inhibits the pathogenicity of the fungus. Thus, the present inventors have discovered that Trehalose-6-Phosphate Synthase is essential for normal rice blast pathogenicity, and can be used as a target for the identification of antibiotics, preferably fungicides. Accordingly, the present invention provides methods for the identification of compounds that inhibit Trehalose-6-Phosphate Synthase expression or activity. The methods of the invention are useful for the identification of antibiotics, preferably fungicides.

### BRIEF DESCRIPTION OF THE FIGURES

FIG. 1 shows the reaction performed by Trehalose-6-Phosphate Synthase (TPS1). The Substrates/Products are UDP-glucose and D-glucose-6-phosphate and the Products/Substrates are UDP and alpha, alpha′-trehalose-6-phosphate. The function of the Trehalose-6-Phosphate Synthase enzyme is the interconversion of UDP-glucose and D-glucose-6-phosphate to UDP and alpha, alpha′-trehalose-6-phosphate. This reaction is part of the trehalose biosynthesis pathway.

FIG. 2 shows a digital image showing the effect of TPS1 gene disruption on Magnaporthe grisea pathogenicity using whole plant infection assays. Rice variety CO39 was inoculated with wild-type and the transposon insertion strains, K1-28 and K1-30. Leaf segments were imaged at five days post-inoculation.

### DETAILED DESCRIPTION OF THE INVENTION

Unless otherwise indicated, the following terms are intended to have the following meanings in interpreting the present invention.

The term “active against” in the context of compounds, agents, or compositions having antibiotic activity indicates that the compound exerts an effect on a particular target or targets which is deleterious to the in vitro and/or in vivo growth of an organism having that target or targets. In particular, a compound active against a gene exerts an action on a target which affects an expression product of that gene. This does not necessarily mean that the compound acts directly on the expression product of the gene, but instead indicates that the compound affects the expression product in a deleterious manner. Thus, the direct target of the compound may be, for example, at an upstream component which reduces transcription from the gene, resulting in a lower level of expression. Likewise, the compound may affect the level of translation of a polypeptide expression product, or may act on a downstream component of a biochemical pathway in which the expression product of the gene has a major biological role. Consequently, such a compound can be said to be active against the gene, against the gene product, or against the related component either upstream or downstream of that gene or expression product. While the term “active against” encompasses a broad range of potential activities, it also implies some degree of specificity of target. Therefore, for example, a general protease is not “active against” a particular gene which produces a polypeptide product. In contrast, a compound which inhibits a particular enzyme is active against that enzyme and against the gene which codes for that enzyme.

As used herein, the term “allele” refers to any of the alternative forms of a gene that may occur at a given locus.

The term “antibiotic” refers to any substance or compound that when contacted with a living cell, organism, virus, or other entity capable of replication, results in a reduction of growth, viability, or pathogenicity of that entity.

The term “antipathogenic,” as used herein, refers to a mutant form of a gene, which inactivates a pathogenic activity of an organism on its host organism or substantially reduces the level of pathogenic activity, wherein “substantially” means a reduction at least as great as the standard deviation for a measurement, preferably a reduction by 50%, more preferably a reduction of at least one magnitude, i.e. to 10%. The pathogenic activity affected may be an aspect of pathogenic activity governed by the normal form of said gene, or the pathway the normal form of said gene functions on, or of the organism's pathogenic activity in general. “Antipathogenic” may also refer to a cell, cells, tissue, or organism that contain said mutant form of a gene; a phenotype associated with said mutant form of a gene, and/or associated with a cell, cells, tissue, or organism that contain said mutant form of a gene.

The term “binding” refers to a non-covalent or a covalent interaction, preferably non-covalent, that holds two molecules together. For example, two such molecules could be an enzyme and an inhibitor of that enzyme. Non-covalent interactions include hydrogen bonding, ionic interactions among charged groups, van der Waals interactions and hydrophobic interactions among nonpolar groups. One or more of these interactions can mediate the binding of two molecules to each other.

The term “biochemical pathway” or “pathway” refers to a connected series of biochemical reactions normally occurring in a cell, or more broadly a cellular event such as cellular division or DNA replication. Typically, the steps in such a biochemical pathway act in a coordinated fashion to produce a specific product or products or to produce some other particular biochemical action. Such a biochemical pathway requires the expression product of a gene if the absence of that expression product either directly or indirectly prevents the completion of one or more steps in that pathway, thereby preventing or significantly reducing the production of one or more normal products or effects of that pathway. Thus, an agent specifically inhibits such a biochemical pathway requiring the expression product of a particular gene if the presence of the agent stops or substantially reduces the completion of the series of steps in that pathway. Such an agent, may, but does not necessarily, act directly on the expression product of that particular gene.

As used herein, the term “conditional lethal” refers to a mutation permitting growth and/or survival only under special growth or environmental conditions.

As used herein, the term “cosmid” refers to a hybrid vector, used in gene cloning, that includes a cos site (from the lambda bacteriophage). It also contains drug resistance marker genes and other plasmid genes. Cosmids are especially suitable for cloning large genes or multigene fragments.

As used herein, the term “dominant allele” refers to a dominant mutant allele in which a discernable mutant phenotype can be detected when this mutation is present in an organism that also contains a wild-type (non-mutant), recessive allele, or other dominant allele.

“Fungi” (singular: fungus) refers to whole fungi, fungal organs and tissues (e.g., asci, hyphae, pseudohyphae, rhizoid, sclerotia, sterigmata, spores, sporodochia, sporangia, synnemata, conidia, ascostroma, cleistothecia, mycelia, perithecia, basidia and the like), spores, fungal cells and the progeny thereof. Fungi are a group of organisms (about 50,000 known species), including, but not limited to, mushrooms, mildews, moulds, yeasts, etc., comprising the kingdom Fungi. They can either exist as single cells or make up a multicellular body called a mycelium, which consists of filaments known as hyphae. Most fungal cells are multinucleate and have cell walls, composed chiefly of chitin. Fungi exist primarily in damp situations on land and, because of the absence of chlorophyll and thus the inability to manufacture their own food by photosynthesis, are either parasites on other organisms or saprotrophs feeding on dead organic matter. The principal criteria used in classification are the nature of the spores produced and the presence or absence of cross walls within the hyphae. Fungi are distributed worldwide in terrestrial, freshwater, and marine habitats. Some live in the soil. Many pathogenic fungi cause disease in animals and man or in plants, while some saprotrophs are destructive to timber, textiles, and other materials. Some fungi form associations with other organisms, most notably with algae to form lichens.

As used herein, the term “fungicide”, “antifungal”, or “antimycotic” refers to an antibiotic substance or compound that kills or suppresses the growth, viability, or pathogenicity of at least one fungus, fungal cell, fungal tissue or spore.

In the context of this disclosure, “gene” should be understood to refer to a unit of heredity. Each gene is composed of a linear chain of deoxyribonucleotides that can be referred to by the sequence of nucleotides forming the chain. Thus, “sequence” is used to indicate both the ordered listing of the nucleotides which form the chain, and the chain, itself, which has that sequence of nucleotides. “Sequence” is used in the similar way in referring to RNA chains, linear chains made of ribonucleotides. The gene may include regulatory and control sequences, sequences that can be transcribed into an RNA molecule, and may contain sequences with unknown function. The majority of the RNA transcription products are messenger RNAs (mRNAs), which include sequences that are translated into polypeptides and may include sequences that are not translated. It should be recognized that small differences in nucleotide sequence for the same gene can exist between different fungal strains, or even within a particular fungal strain, without altering the identity of the gene.

As used in this disclosure, the terms “growth” or “cell growth” of an organism refers to an increase in mass, density, or number of cells of said organism. Some common methods for the measurement of growth include the determination of the optical density of a cell suspension, the counting of the number of cells in a fixed volume, the counting of the number of cells by measurement of cell division, the measurement of cellular mass or cellular volume, and the like.

As used in this disclosure, the term “growth conditional phenotype” indicates that a fungal strain having such a phenotype exhibits a significantly greater difference in growth rates in response to a change in one or more of the culture parameters than an otherwise similar strain not having a growth conditional phenotype. Typically, a growth conditional phenotype is described with respect to a single growth culture parameter, such as temperature. Thus, a temperature (or heat-sensitive) mutant (i.e., a fungal strain having a heat-sensitive phenotype) exhibits significantly different growth, and preferably no growth, under non-permissive temperature conditions as compared to growth under permissive conditions. In addition, such mutants preferably also show intermediate growth rates at intermediate, or semi-permissive, temperatures. Similar responses also result from the appropriate growth changes for other types of growth conditional phenotypes.

As used herein, the term “heterologous TPS1 gene” means a gene, not derived from Magnaporthe grisea, and having: at least 50% sequence identity, preferably 60%, 70%, 80%, 90%, 95%, 99% sequence identity and each integer unit of sequence identity from 50-100% in ascending order to SEQ ID NO: 1 or SEQ ID NO: 2; or at least 10% of the activity of a Magnaporthe grisea Trehalose-6-Phosphate Synthase, preferably 25%, 50%, 75%, 90%, 95%, 99% and each integer unit of activity from 10-100% in ascending order.

As used herein, the term “His-Tag” refers to an encoded polypeptide consisting of multiple consecutive histidine amino acids.

As used herein, the terms “hph,” “hygromycin B phosphotransferase,” and “hygromycin resistance gene” refer to the E. coli hygromycin phosphotransferase gene or gene product.

As used herein, the term “hygromycin B” refers to an aminoglycosidic antibiotic, used for selection and maintenance of eukaryotic cells containing the E. coli hygromycin resistance gene.

As used herein, the term “imperfect state” refers to a classification of a fungal organism having no demonstrable sexual life stage.

The term “inhibitor,” as used herein, refers to a chemical substance that inactivates the enzymatic activity of Trehalose-6-Phosphate Synthase or substantially reduces the level of enzymatic activity, wherein “substantially” means a reduction at least as great as the standard deviation for a measurement, preferably a reduction by 50%, more preferably a reduction of at least one magnitude, i.e. to 10%. The inhibitor may function by interacting directly with the enzyme, a cofactor of the enzyme, the substrate of the enzyme, or any combination thereof.

A polynucleotide may be “introduced” into a fungal cell by any means known to those of skill in the art, including transfection, transformation or transduction, transposable element, electroporation, particle bombardment, infection and the like. The introduced polynucleotide may be maintained in the cell stably if it is incorporated into a non-chromosomal autonomous replicon or integrated into the fungal chromosome. Alternatively, the introduced polynucleotide may be present on an extra-chromosomal non-replicating vector and be transiently expressed or transiently active.

As used herein, the term “knockout” or “gene disruption” refers to the creation of organisms carrying a null mutation (a mutation in which there is no active gene product), a partial null mutation or mutations, or an alteration or alterations in gene regulation by interrupting a DNA sequence through insertion of a foreign piece of DNA. Usually the foreign DNA encodes a selectable marker.

The term “method of screening” means that the method is suitable, and is typically used, for testing for a particular property or effect in a large number of compounds. Typically, more than one compound is tested simultaneously (as in a 96-well microtiter plate), and preferably significant portions of the procedure can be automated. Method of screening also refers to the determination of a set of different properties or effects of one compound simultaneously.

As used herein, the term “mutant form” of a gene refers to a gene which has been altered, either naturally or artificially, changing the base sequence of the gene. The change in the base sequence may be of several different types, including changes of one or more bases for different bases, deletions, and/or insertions, such as by a transposon. In contrast, a normal form of a gene (wild-type) is a form commonly found in natural populations of an organism. Commonly a single form of a gene will predominate in natural populations. In general, such a gene is suitable as a normal form of a gene, however, other forms which provide similar functional characteristics may also be used as a normal gene. In particular, a normal form of a gene does not confer a growth conditional phenotype on the strain having that gene, while a mutant form of a gene suitable for use in these methods does provide such a growth conditional phenotype.

As used herein, the term “Ni-NTA” refers to nickel sepharose.

As used herein, a “normal” form of a gene (wild-type) is a form commonly found in natural populations of an organism. Commonly a single form of a gene will predominate in natural populations. In general, such a gene is suitable as a normal form of a gene, however, other forms which provide similar functional characteristics may also be used as a normal gene. In particular, a normal form of a gene does not confer a growth conditional phenotype on the strain having that gene, while a mutant form of a gene suitable for use in these methods does provide such a growth conditional phenotype.

As used herein, the term “one form” of a gene is synonymous with the term “gene,” and a “different form” of a gene refers to a gene that has greater than 49% sequence identity and less than 100% sequence identity with said first form.

As used herein, the term “pathogenicity” refers to a capability of causing disease and/or degree of capacity to cause disease. The term is applied to parasitic microorganisms in relation to their hosts. As used herein, “pathogenicity,” “pathogenic,” and the like, encompass the general capability of causing disease as well as various mechanisms and structural and/or functional deviations from normal used in the art to describe the causative factors and/or mechanisms, presence, pathology, and/or progress of disease, such as virulence, host recognition, cell wall degradation, toxin production, infection hyphae, penetration peg production, appressorium production, lesion formation, sporulation, and the like.

The “percent (%) sequence identity” between two polynucleotide or two polypeptide sequences is determined according to the either the BLAST program (Basic Local Alignment Search Tool; (Altschul, S. F. et al., 215 J. Mol. Biol. 403 (1990)) or using Smith Waterman Alignment (T. F. Smith & M. S. Waterman (1981) 147 J. Mol. Biol. 195 (1981)) as incorporated into GENEMATCHER PLUS. It is understood that for the purposes of determining sequence identity when comparing a DNA sequence to an RNA sequence, a thymine nucleotide is equivalent to a uracil nucleotide.

By “polypeptide” is meant a chain of at least two amino acids joined by peptide bonds. The chain may be linear, branched, circular or combinations thereof. Preferably, polypeptides are from about 10 to about 1000 amino acids in length, more preferably 10-50 amino acids in length. The polypeptides may contain amino acid analogs and other modifications, including, but not limited to glycosylated or phosphorylated residues.

As used herein, the term “proliferation” is synonymous to the term “growth.”

As used herein, “semi-permissive conditions” are conditions in which the relevant culture parameter for a particular growth conditional phenotype is intermediate between permissive conditions and non-permissive conditions. Consequently, in semi-permissive conditions an organism having a growth conditional phenotype will exhibit growth rates intermediate between those shown in permissive conditions and non-permissive conditions. In general, such intermediate growth rate may be due to a mutant cellular component which is partially functional under semi-permissive conditions, essentially fully functional under permissive conditions, and is non-functional or has very low function under non-permissive conditions, where the level of function of that component is related to the growth rate of the organism. An intermediate growth rate may also be a result of a nutrient substance or substances that are present in amounts not sufficient for optimal growth rates to be achieved.

“Sensitivity phenotype” refers to a phenotype that exhibits either hypersensitivity or hyposensitivity.

The term “specific binding” refers to an interaction between Trehalose-6-Phosphate Synthase and a molecule or compound, wherein the interaction is dependent upon the primary amino acid sequence and/or the conformation of Trehalose-6-Phosphate Synthase.

As used herein, the term “TPS1” means a gene encoding Trehalose-6-Phosphate Synthase activity, referring to an enzyme that catalyses the interconversion of UDP-glucose and D-glucose-6-phosphate with UDP and alpha, alpha′-trehalose-6-phosphate, and may also be used to refer to the gene product.

“Transform,” as used herein, refers to the introduction of a polynucleotide (single or double stranded DNA, RNA, or a combination thereof) into a living cell by any means. Transformation may be accomplished by a variety of methods, including, but not limited to, electroporation, polyethylene glycol mediated uptake, particle bombardment, agrotransformation, and the like. This process may result in transient or stable expression of the transformed polynucleotide. By “stably transformed” is meant that the sequence of interest is integrated into a replicon in the cell, such as a chromosome or episome. Transformed cells encompass not only the end product of a transformation process, but also the progeny thereof which retain the polynucleotide of interest.

For the purposes of the invention, “transgenic” refers to any cell, spore, tissue or part, that contains all or part of at least one recombinant polynucleotide. In many cases, all or part of the recombinant polynucleotide is stably integrated into a chromosome or stable extra-chromosomal element, so that it is passed on to successive generations.

As used herein, the terms “Trehalose-6-Phosphate Synthase” and “Trehalose-6-Phosphate Synthase polypeptide” are synonymous with “the TPS1 gene product” and refer to an enzyme that catalyses the interconversion of UDP-glucose and D-glucose-6-phosphate with UDP and alpha, alpha′-trehalose-6-phosphate.

As used herein, the term “Tween 20” means sorbitan mono-9-octadecenoate poly (oxy-1,1-ethanediyl).

As used in this disclosure, the term “viability” of an organism refers to the ability of an organism to demonstrate growth under conditions appropriate for said organism, or to demonstrate an active cellular function. Some examples of active cellular functions include respiration as measured by gas evolution, secretion of proteins and/or other compounds, dye exclusion, mobility, dye oxidation, dye reduction, pigment production, changes in medium acidity, and the like.

The present inventors have discovered that disruption of the TPS1 gene and/or gene product inhibits the pathogenicity of Magnaporthe grisea. Thus, the inventors are the first to demonstrate that Trehalose-6-Phosphate Synthase is a target for antibiotics, preferably antifungals.

Accordingly, the invention provides methods for identifying compounds that inhibit TPS1 gene expression or biological activity of its gene product(s). Such methods include ligand-binding assays, assays for enzyme activity, cell-based assays, and assays for TPS1 gene expression. Any compound that is a ligand for Trehalose-6-Phosphate Synthase may have antibiotic activity. For the purposes of the invention, “ligand” refers to a molecule that will bind to a site on a polypeptide. The compounds identified by the methods of the invention are useful as antibiotics.

Thus, in one embodiment, the invention provides a method for identifying a test compound as a candidate for an antibiotic, comprising contacting a Trehalose-6-Phosphate Synthase polypeptide with a test compound and detecting the presence or absence of binding between the test compound and the Trehalose-6-Phosphate Synthase polypeptide, wherein binding indicates that the test compound is a candidate for an antibiotic.

The Trehalose-6-Phosphate Synthase protein may have the amino acid sequence of a naturally occurring Trehalose-6-Phosphate Synthase found in a fungus, animal, plant, or microorganism, or may have an amino acid sequence derived from a naturally occurring sequence. Preferably the Trehalose-6-Phosphate Synthase is a fungal Trehalose-6-Phosphate Synthase. The cDNA (SEQ ID NO: 1) encoding the Trehalose-6-Phosphate Synthase protein, the genomic DNA (SEQ ID NO: 2) encoding the M. grisea protein, and the polypeptide (SEQ ID NO: 3) can be found herein.

In an alternate embodiment, the invention provides for a polypeptide consisting essentially of SEQ ID NO: 3. For the purposes of the present invention, a polypeptide consisting essentially of SEQ ID NO: 3 has at least 80% sequence identity with SEQ ID NO: 3 and catalyses the interconversion of UDP-glucose and D-glucose-6-phosphate with UDP and alpha, alpha′-trehalose-6-phosphate with at least 10% of the activity of SEQ ID NO: 3. Preferably, the polypeptide consisting essentially of SEQ ID NO: 3 has at least 85% sequence identity with SEQ ID NO: 3, more preferably the sequence identity is at least 90%, most preferably the sequence identity is at least 95% or 97 or 99%, or any integer from 80-100% sequence identity in ascending order. Preferably, the polypeptide consisting essentially of SEQ ID NO: 3 has at least 25%, at least 50%, at least 75% or at least 90% of the activity of M. grisea Trehalose-6-Phosphate Synthase, or any integer from 60-100% activity in ascending order.

By “fungal Trehalose-6-Phosphate Synthase” is meant an enzyme that can be found in at least one fungus, and which catalyzes the interconversion of of UDP-glucose and D-glucose-6-phosphate with UDP and alpha, alpha′-trehalose-6-phosphate. The Trehalose-6-Phosphate Synthase may be from any of the fungi, including ascomycota, zygomycota, basidiomycota, chytridiomycota, and lichens.

In one embodiment, the Trehalose-6-Phosphate Synthase is a Magnaporthe Trehalose-6-Phosphate Synthase. Magnaporthe species include, but are not limited to, Magnaporthe rhizophila, Magnaporthe salvinii, Magnaporthe grisea and Magnaporthe poae and the imperfect states of Magnaporthe in the genus Pyricularia. Preferably, the Magnaporthe Trehalose-6-Phosphate Synthase is from Magnaporthe grisea.

In various embodiments, the Trehalose-6-Phosphate Synthase can be from Powdery Scab (Spongospora subterranea), Grey Mould (Botrytis cinerea), White Rot (Armillaria mellea), Heartrot Fungus (Ganoderma adspersum), Brown-Rot (Piptoporus betulinus), Corn Smut (Ustilago maydis), Heartrot (Polyporus squamosus), Gray Leaf Spot (Cercospora zeae-maydis), Honey Fungus (Armillaria gallica), Root rot (Armillaria luteobubalina), Shoestring Rot (Armillaria ostoyae), Banana Anthracnose Fungus (Colletotrichum musae), Apple-rotting Fungus (Monilinia fructigena), Apple-rotting Fungus (Penicillium expansum), Clubroot Disease (Plasmodiophora brassicae), Potato Blight (Phytophthora infestans), Root pathogen (Heterobasidion annosum), Take-all Fungus (Gaeumannomyces graminis), Dutch Elm Disease (Ophiostoma ulmi), Bean Rust (Uromyces appendiculatus), Northern Leaf Spot (Cochliobolus carbonum), Milo Disease (Periconia circinata), Southern Corn Blight (Cochliobolus heterostrophus), Leaf Spot (Cochliobolus lunata), Brown Stripe (Cochliobolus stenospilus), Panama disease (Fusarium oxysporum), Wheat Head Scab Fungus (Fusarium graminearum), Cereal Foot Rot (Fusarium culmorum), Potato Black Scurf (Rhizoctonia solani), Wheat Black Stem Rust (Puccinia graminis), White mold (Sclerotinia sclerotiorum), and the like.

Fragments of a Trehalose-6-Phosphate Synthase polypeptide may be used in the methods of the invention, preferably if the fragments include an intact or nearly intact epitope that occurs on the biologically active wild-type Trehalose-6-Phosphate Synthase. The fragments comprise at least 10 consecutive amino acids of a Trehalose-6-Phosphate Synthase. Preferably, the fragment comprises at least 15, 20, 25, 30, 35, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, or at least 520 consecutive amino acids residues of an Trehalose-6-Phosphate Synthase. In one embodiment, the fragment is from a Magnaporthe Trehalose-6-Phosphate Synthase. Preferably, the fragment contains an amino acid sequence conserved among fungal Trehalose-6-Phosphate Synthases.

Polypeptides having at least 50% sequence identity with a fungal Trehalose-6-Phosphate Synthase are also useful in the methods of the invention. Preferably, the sequence identity is at least 60%, more preferably the sequence identity is at least 70%, most preferably the sequence identity is at least 80% or 90 or 95 or 99%, or any integer from 60-100% sequence identity in ascending order.

In addition, it is preferred that the polypeptide has at least 10% of the activity of a fungal Trehalose-6-Phosphate Synthase. More preferably, the polypeptide has at least 25%, at least 50%, at least 75% or at least 90% of the activity of a fungal Trehalose-6-Phosphate Synthase. Most preferably, the polypeptide has at least 10%, at least 25%, at least 50%, at least 75% or at least 90% of the activity of the M. grisea Trehalose-6-Phosphate Synthase protein.

Thus, in another embodiment, the invention provides a method for identifying a test compound as a candidate for a fungicide, comprising: contacting a test compound with at least one polypeptide selected from the group consisting of: a polypeptide having at least ten consecutive amino acids of a fungal Trehalose-6-Phosphate Synthase, a polypeptide having at least 50% sequence identity with a fungal Trehalose-6-Phosphate Synthase, and a polypeptide having at least 10% of the activity of a fungal Trehalose-6-Phosphate Synthase; and detecting the presence and/or absence of binding between said test compound and said polypeptide, wherein binding indicates that said test compound is a candidate for an antibiotic.

Any technique for detecting the binding of a ligand to its target may be used in the methods of the invention. For example, the ligand and target are combined in a buffer. Many methods for detecting the binding of a ligand to its target are known in the art, and include, but are not limited to, the detection of an immobilized ligand-target complex or the detection of a change in the properties of a target when it is bound to a ligand. For example, in one embodiment, an array of immobilized candidate ligands is provided. The immobilized ligands are contacted with a Trehalose-6-Phosphate Synthase protein or a fragment or variant thereof, the unbound protein is removed and the bound Trehalose-6-Phosphate Synthase is detected. In a preferred embodiment, bound Trehalose-6-Phosphate Synthase is detected using a labeled binding partner, such as a labeled antibody. In an alternate preferred embodiment, Trehalose-6-Phosphate Synthase is labeled prior to contacting the immobilized candidate ligands. Preferred labels include fluorescent or radioactive moieties. Preferred detection methods include fluorescence correlation spectroscopy (FCS) and FCS-related confocal nanofluorimetric methods.

Once a compound is identified as a candidate for an antibiotic, it can be tested for the ability to inhibit Trehalose-6-Phosphate Synthase enzymatic activity. The compounds can be tested using either in vitro or cell based assays. Alternatively, a compound can be tested by applying it directly to a fungus or fungal cell, or expressing it therein, and monitoring the fungus or fungal cell for changes or decreases in growth, development, viability, pathogenicity, or alterations in gene expression. Thus, in one embodiment, the invention provides a method for determining whether a compound identified as an antibiotic candidate by an above method has antifungal activity, further comprising: contacting a fungus or fungal cells with said antifungal candidate and detecting a decrease in the growth, viability, or pathogenicity of said fungus or fungal cells.

By decrease in growth, is meant that the antifungal candidate causes at least a 10% decrease in the growth of the fungus or fungal cells, as compared to the growth of the fungus or fungal cells in the absence of the antifungal candidate. By a decrease in viability is meant that at least 20% of the fungal cells, or portion of the fungus contacted with the antifungal candidate are nonviable. Preferably, the growth or viability will be decreased by at least 40%. More preferably, the growth or viability will be decreased by at least 50%, 75% or at least 90% or more. Methods for measuring fungal growth and cell viability are known to those skilled in the art. By decrease in pathogenicity, is meant that the antifingal candidate causes at least a 10% decrease in the disease caused by contact of the fungal pathogen with its host, as compared to the disease caused in the absence of the antifungal candidate. Preferably, the disease will be decreased by at least 40%. More preferably, the disease will be decreased by at least 50%, 75% or at least 90% or more. Methods for measuring fungal disease are well known to those skilled in the art, and include such metrics as lesion formation, lesion size, sporulation, respiratory failure, and/or death.

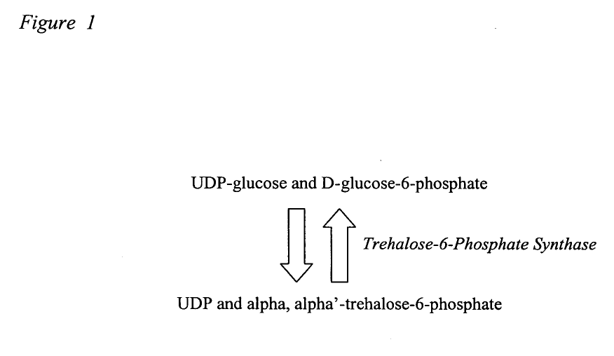

The ability of a compound to inhibit Trehalose-6-Phosphate Synthase activity can be detected using in vitro enzymatic assays in which the disappearance of a substrate or the appearance of a product is directly or indirectly detected. Trehalose-6-Phosphate Synthase catalyzes the irreversible or reversible reaction UDP-glucose and D-glucose-6-phosphate=UDP and alpha, alpha′-trehalose-6-phosphate (see FIG. 1). Methods for detection of UDP, UDP-glucose, D-glucose-6-phosphate, and/or alpha, alpha′-trehalose-6-phosphate, include spectrophotometry, mass spectroscopy, thin layer chromatography (TLC) and reverse phase HPLC.

Thus, the invention provides a method for identifying a test compound as a candidate for an antibiotic, comprising: contacting UDP-glucose and D-glucose-6-phosphate with a Trehalose-6-Phosphate Synthase; contacting UDP-glucose and D-glucose-6-phosphate with Trehalose-6-Phosphate Synthase and a test compound; and determining the change in concentration for at least one of the following: UDP, UDP-glucose, D-glucose-6-phosphate, and/or alpha, alpha′-trehalose-6-phosphate, wherein a change in concentration for any of the above substances indicates that said test compound is a candidate for an antibiotic.

An alternate embodiment the present invention is a method for identifying a test compound as a candidate for an antibiotic, comprising: contacting UDP and alpha, alpha′-trehalose-6-phosphate with a Trehalose-6-Phosphate Synthase; contacting UDP and alpha, alpha′-trehalose-6-phosphate with a Trehalose-6-Phosphate Synthase and a test compound; and determining the change in concentration for at least one of the following: UDP, UDP-glucose, D-glucose-6-phosphate, and/or alpha, alpha′-trehalose-6-phosphate, wherein a change in concentration for any of the above substances indicates that said test compound is a candidate for an antibiotic.

Enzymatically active fragments of a fungal Trehalose-6-Phosphate Synthase are also useful in the methods of the invention. For example, an enzymatically active polypeptide comprising at least 100 consecutive amino acid residues of a fungal Trehalose-6-Phosphate Synthase may be used in the methods of the invention. In addition, an enzymatically active polypeptide having at least 50%, 60%, 70%, 80%, 90%, 95% or at least 98% sequence identity with a fungal Trehalose-6-Phosphate Synthase may be used in the methods of the invention. Most preferably, the polypeptide has at least 50% sequence identity with a fungal Trehalose-6-Phosphate Synthase and at least 10%, 25%, 75% or at least 90% of the activity thereof.

Thus, the invention provides a method for identifying a test compound as a candidate for an antibiotic, comprising: contacting UDP-glucose and D-glucose-6-phosphate with a polypeptide selected from the group consisting of: a polypeptide having at least 50% sequence identity with a Trehalose-6-Phosphate Synthase, a polypeptide having at least 50% sequence identity with a Trehalose-6-Phosphate Synthase and having at least 10% of the activity thereof, and a polypeptide comprising at least 100 consecutive amino acids of a Trehalose-6-Phosphate Synthase; contacting UDP-glucose and D-glucose-6-phosphate with said polypeptide and a test compound; and determining the change in concentration for at least one of the following: UDP, UDP-glucose, D-glucose-6-phosphate, and/or alpha, alpha′-trehalose-6-phosphate, wherein a change in concentration for any of the above substances indicates that said test compound is a candidate for an antibiotic.

An alternate embodiment the present invention is a method for identifying a test compound as a candidate for an antibiotic, comprising: contacting UDP and alpha, alpha′-trehalose-6-phosphate with a polypeptide selected from the group consisting of: a polypeptide having at least 50% sequence identity with a Trehalose-6-Phosphate Synthase, a polypeptide having at least 50% sequence identity with a Trehalose-6-Phosphate Synthase and at least 10% of the activity thereof, and a polypeptide comprising at least 100 consecutive amino acids of a Trehalose-6-Phosphate Synthase; contacting UDP and alpha, alpha′-trehalose-6-phosphate, with said polypeptide and a test compound; and determining the change in concentration for at least one of the following, UDP, UDP-glucose, D-glucose-6-phosphate, and/or alpha, alpha′-trehalose-6-phosphate, wherein a change in concentration for any of the above substances indicates that said test compound is a candidate for an antibiotic.

For the in vitro enzymatic assays, Trehalose-6-Phosphate Synthase protein and derivatives thereof may be purified from a fungus or may be recombinantly produced in and purified from an archael, bacterial, fungal, or other eukaryotic cell culture. Preferably these proteins are produced using an E. coli, yeast, or filamentous fungal expression system. Methods for the purification Trehalose-6-Phosphate Synthase may be described in Vandercammen et al. 182 Eur. J. Biochem. 613-620 (1989). Other methods for the purification of Trehalose-6-Phosphate Synthase proteins and polypeptides are known to those skilled in the art.

As an alternative to in vitro assays, the invention also provides cell based assays. In one embodiment, the invention provides a method for identifying a test compound as a candidate for an antibiotic, comprising: measuring the expression of a Trehalose-6-Phosphate Synthase in a cell, cells, tissue, or an organism in the absence of a test compound; contacting said cell, cells, tissue, or organism with said test compound and measuring the expression of said Trehalose-6-Phosphate Synthase in said cell, cells, tissue, or organism; and comparing the expression of Trehalose-6-Phosphate Synthase in steps (a) and (b), wherein a lower expression in the presence of said test compound indicates that said compound is a candidate for an antibiotic.

Expression of Trehalose-6-Phosphate Synthase can be measured by detecting the TPS1 primary transcript or mRNA, Trehalose-6-Phosphate Synthase polypeptide, or Trehalose-6-Phosphate Synthase enzymatic activity. Methods for detecting the expression of RNA and proteins are known to those skilled in the art. (See, e.g., Current Protocols in Molecular Biology, Ausubel et al., eds., Greene Publishing & Wiley-Interscience, New York, (1995)). The method of detection is not critical to the present invention. Methods for detecting TPS1 RNA include, but are not limited to amplification assays such as quantitative reverse transcriptase-PCR, and/or hybridization assays such as Northern analysis, dot blots, slot blots, in-situ hybridization, transcriptional fusions using a TPS1 promoter fused to a reporter gene, DNA assays, and microarray assays.

Methods for detecting protein expression include, but are not limited to, immunodetection methods such as Western blots, ELISA assays, polyacrylamide gel electrophoresis, mass spectroscopy, and enzymatic assays. Also, any reporter gene system may be used to detect TPS1 protein expression. For detection using gene reporter systems, a polynucleotide encoding a reporter protein is fused in frame with TPS1, so as to produce a chimeric polypeptide. Methods for using reporter systems are known to those skilled in the art.

Chemicals, compounds or compositions identified by the above methods as modulators, preferably inhibitors, of TPS1 expression or activity can then be used to control fungal growth. Diseases such as rusts, mildews, and blights spread rapidly once established. Fungicides are thus routinely applied to growing and stored crops as a preventive measure, generally as foliar sprays or seed dressings. For example, compounds that inhibit fungal growth can be applied to a fungus or expressed in a fungus, in order to prevent fungal growth. Thus, the invention provides a method for inhibiting fungal growth, comprising contacting a fungus with a compound identified by the methods of the invention as having antifungal activity.

Antifungals and antifungal inhibitor candidates identified by the methods of the invention can be used to control the growth of undesired fungi, including ascomycota, zygomycota, basidiomycota, chytridiomycota, and lichens.

Examples of undesired fungi include, but are not limited to Powdery Scab (Spongospora subterranea), Grey Mould (Botrytis cinerea), White Rot (Armillaria mellea), Heartrot Fungus (Ganoderma adspersum), Brown-Rot (Piptoporus betulinus), Corn Smut (Ustilago maydis), Heartrot (Polyporus squamosus), Gray Leaf Spot (Cercospora zeae-maydis), Honey Fungus (Armillaria gallica), Root rot (Armillaria luteobubalina), Shoestring Rot (Armillaria ostoyae), Banana Anthracnose Fungus (Colletotrichum musae), Apple-rotting Fungus (Monilinia fructigena), Apple-rotting Fungus (Penicillium expansum), Clubroot Disease (Plasmodiophora brassicae), Potato Blight (Phytophthora infestans), Root pathogen (Heterobasidion annosum), Take-all Fungus (Gaeumannomyces graminis), Dutch Elm Disease (Ophiostoma ulmi), Bean Rust (Uromyces appendiculatus), Northern Leaf Spot (Cochliobolus carbonum), Milo Disease (Periconia circinata), Southern Corn Blight (Cochliobolus heterostrophus), Leaf Spot (Cochliobolus lunata), Brown Stripe (Cochliobolus stenospilus), Panama disease (Fusarium oxysporum), Wheat Head Scab Fungus (Fusarium graminearum), Cereal Foot Rot (Fusarium culmorum), Potato Black Scurf (Rhizoctonia solani), Wheat Black Stem Rust (Puccinia graminis), White mold (Sclerotinia sclerotiorum), diseases of animals such as infections of lungs, blood, brain, skin, scalp, nails or other tissues (Aspergillus fumigatus Aspergillus sp. Fusraium sp., Trichophyton sp., Epidermophyton sp., and Microsporum sp., and the like).

Also provided is a method of screening for an antibiotic by determining whether a test compound is active against the gene identified (SEQ ID NO: 1 or SEQ ID NO: 2), its gene product (SEQ ID NO: 3), or the biochemical pathway or pathways on which it functions.

In a prefered alternate embodiment, the method of the present invention is performed by providing an organism having a first form of the gene corresponding to either SEQ ID NO: 1 or SEQ ID NO: 2, either a normal form, a mutant form, a homologue, or a heterologous TPS1 gene that performs a similar function as TPS1. The first form of TPS1 may or may not confer a growth conditional phenotype, i.e., a trehalose requiring phenotype, and/or a hypersensitivity or hyposensitivity phenotype on the organism having that altered form. In one particular embodiment a mutant form contains a transposon insertion. A comparison organism having a second form of a TPS1, different from the first form of the gene is also provided, and the two organisms are separately contacted with a test compound. The growth of the two organisms in the presence of the test compound is then compared.

Thus, in one embodiment, the invention provides a method for identifying a test compound as a candidate for an antibiotic, comprising: providing cells having one form of a Trehalose-6-Phosphate Synthase gene, and providing comparison cells having a different form of a Trehalose-6-Phosphate Synthase gene; and contacting said cells and said comparison cells with a test compound and determining the growth of said cells and said comparison cells in the presence of the test compound, wherein a difference in growth between said cells and said comparison cells in the presence of said test compound indicates that said test compound is a candidate for an antibiotic.

It is recognized in the art that the optional determination of the growth of said first organism and said comparison second organism in the absence of any test compounds may be performed to control for any inherent differences in growth as a result of the different genes. It is also recognized that any combination of two different forms of a TPS1 gene, including normal genes, mutant genes, homologues, and functional homologues may be used in this method. Growth and/or proliferation of an organism is measured by methods well known in the art such as optical density measurements, and the like. In a preferred embodiment the organism is Magnaporthe grisea.

In an alternate embodiment, the method is performed by providing an organism having a first form of the gene corresponding to either SEQ ID NO: 1 or SEQ ID NO: 2, either a normal form, a mutant form, a homologue, or a heterologous TPS1 gene that performs a similar function as TPS1. The first form of TPS1 may or may not confer an antipathogenic phenotype. In one particular embodiment a mutant form contains a transposon insertion. A comparison organism having a second form of a TPS1, different from the first form of the gene is also provided, and the two organisms are separately contacted with a test compound. The pathogenicity of the two organisms in the presence of the test compound is then compared.

Thus, in one embodiment, the invention provides a method for identifying a test compound as a candidate for an antibiotic, comprising: providing cells having one form of a Trehalose-6-Phosphate Synthase gene, and providing comparison cells having a different form of a Trehalose-6-Phosphate Synthase gene; and contacting said cells and said comparison cells with a test compound and determining the pathogenicity of said cells and said comparison cells in the presence of the test compound, wherein a difference in pathogenicity between said cells and said comparison cells in the presence of said test compound indicates that said test compound is a candidate for an antibiotic.

It is recognized in the art that the optional determination of the pathogenicity of said first organism and said comparison second organism in the absence of any test compounds may be performed to control for any inherent differences in pathogenicity as a result of the different genes. It is also recognized that any combination of two different forms of a TPS1 gene, including normal genes, mutant genes, homologues, and functional homologues may be used in this method. Pathogenicity of an organism is measured by methods well known in the art such as lesion number, lesion size, sporulation, and the like. In a preferred embodiment the organism is Magnaporthe grisea.

Conditional lethal mutants may identify particular biochemical and/or genetic pathways given that at least one identified target gene is present in that pathway. Knowledge of these pathways allows for the screening of test compounds as candidates for antibiotics as inhibitors of the substrates, products and enzymes of the pathway. Pathways known in the art may be found at the Kyoto Encyclopedia of Genes and Genomes and in standard biochemistry texts (See, e.g. Lehninger et al., Principles of Biochemistry, New York, Worth Publishers (1993)).

Thus, in one embodiment, the invention provides a method for screening for test compounds acting against the biochemical and/or genetic pathway or pathways in which TPS1 functions, comprising: providing cells having one form of a gene in the trehalose biochemical and/or genetic pathway and providing comparison cells having a different form of said gene; contacting said cells and said comparison cells with a test compound; and determining the growth of said cells and said comparison cells in the presence of said test compound, wherein a difference in growth between said cells and said comparison cells in the presence of said test compound indicates that said test compound is a candidate for an antibiotic.

The use of multi-well plates for screening is a format that readily accommodates multiple different assays to characterize various compounds, concentrations of compounds, and fungal strains in varying combinations and formats. Certain testing parameters for the screening method can significantly affect the identification of growth inhibitors, and thus can be manipulated to optimize screening efficiency and/or reliability. Notable among these factors are variable sensitivities of different mutants, increasing hypersensitivity with increasingly less permissive conditions, an apparent increase in hypersensitivity with increasing compound concentration, and other factors known to those in the art.

Antipathogenic mutants may identify particular biochemical and/or genetic pathways given that at least one identified target gene is present in that pathway. Knowledge of these pathways allows for the screening of test compounds as candidates for antibiotics as inhibitors of the substrates, products and enzymes of the pathway. Pathways known in the art may be found at the Kyoto Encyclopedia of Genes and Genomes and in standard biochemistry texts (e.g. Lehninger et al., supra).

Thus, in an alternate embodiment, the invention provides a method for screening for test compounds acting against the biochemical and/or genetic pathway or pathways in which TPS1 functions, comprising: providing cells having one form of a gene in the trehalose biochemical and/or genetic pathway and providing comparison cells having a different form of said gene; contacting said cells and said comparison cells with a test compound; and determining the pathogenicity of said cells and said comparison cells in the presence of said test compound, wherein a difference in pathogenicity between said cells and said comparison cells in the presence of said test compound indicates that said test compound is a candidate for an antibiotic.

The use of multi-well plates for screening is a format that readily accommodates multiple different assays to characterize various compounds, concentrations of compounds, and fungal strains in varying combinations and formats. Certain testing parameters for the screening method can significantly affect the identification of pathogenicity inhibitors, and thus can be manipulated to optimize screening efficiency and/or reliability. Notable among these factors are variable sensitivities of different mutants, an apparent increase in sensitivity with increasing compound concentration, and other factors known to those in the art.

### EXPERIMENTAL

### EXAMPLE 1

### Construction of Plasmids with a Transposon Containing a Selectable Marker

Construction of Sif Transposon

Sif was constructed using the GPS3 vector from the GPS-M mutagenesis system from New England Biolabs, Inc. (Beverly, Mass.) as a backbone. This system is based on the bacterial transposon Tn7. The following manipulations were done to GPS3 according to Sambrook et al., Molecular Cloning, a Laboratory Manual, Cold Spring Harbor Laboratory Press (1989). The kanamycin resistance gene (npt) contained between the Tn7 arms was removed by EcoRV digestion. The bacterial hygromycin B phosphotransferase (hph) gene (Gritz & Davies, 25 Gene 179 (1983)) under control of the Aspergillus nidulans trpC promoter and terminator (Mullaney et al., 199 Mol. Gen. Genet. 37 (1985)) was cloned by a HpaI/EcoRV blunt ligation into the Tn7 arms of the GPS3 vector yielding pSif1. Excision of the ampicillin resistance gene (bla) from pSif1 was achieved by cutting pSif1 with XmnI and BglI followed by a T4 DNA polymerase treatment to remove the 3′ overhangs left by the BglI digestion and religation of the plasmid to yield pSif. Top 10F′ electrocompetent E. coli cells (Invitrogen) were transformed with ligation mixture according to manufacturer's recommendations. Transformants containing the Sif transposon were selected on LB agar (Sambrook et al., supra) containing 50 ug/ml of hygromycin B (Sigma Chem. Co., St. Louis, Mo.).

### EXAMPLE 2

Construction of a Fungal Cosmid Library Cosmid libraries were constructed in the pcosKA5 vector (Hamer et al., 98 Proc. Nat'l. Acad. Sci. USA 5110 (2001) (PMID: 11296265)) as described in Sambrook et al. Cosmid libraries were quality checked by pulsed-field gel electrophoresis, restriction digestion analysis, and PCR identification of single genes.

### EXAMPLE 3

### Construction of Cosmids with Transposon Insertion into Fungal Genes

Sif Transposition into a Cosmid

Transposition of Sif into the cosmid framework was carried out as described by the GPS-M mutagenesis system (New England Biolabs, Inc.). Briefly, 2 ul of the 10×GPS buffer, 70 ng of supercoiled pSIF, 8-12 ug of target cosmid DNA were mixed and taken to a final volume of 20 ul with water. 1 ul of transposase (TnsABC) was added to the reaction and incubated for 10 minutes at 37° C. to allow the assembly reaction to occur. After the assembly reaction, 1 ul of start solution was added to the tube, mixed well, and incubated for 1 hour at 37° C. followed by heat inactivation of the proteins at 75° C. for 10 minutes. Destruction of the remaining untransposed pSif was completed by PISceI digestion at 37° C. for 2 hours followed by a 10 minute incubation at 75° C. to inactivate the proteins. Transformation of Top10F′ electrocompetent cells (Invitrogen) was done according to manufacturers recommendations. Sif-containing cosmid transformants were selected by growth on LB agar plates containing 50 ug/ml of hygromycin B (Sigma Chem. Co.) and 100 ug/ml of Ampicillin (Sigma Chem. Co.).

### EXAMPLE 4

### High Throughput Preparation and Verification of Transposon Insertion into the M. grisea TPS1 Gene

E. coli strains containing cosmids with transposon insertions were picked to 96 well growth blocks (Beckman Co.) containing 1.5 ml of TB (Terrific Broth, Sambrook et al., supra) supplemented with 50 ug/ml of ampicillin. Blocks were incubated with shaking at 37° C. overnight. E. coli cells were pelleted by centrifugation and cosmids were isolated by a modified alkaline lysis method (Marra et al., 7 Genome Res. 1072 (1997)). DNA quality was checked by electrophoresis on agarose gels. Cosmids were sequenced using primers from the ends of each transposon and commercial dideoxy sequencing kits (Big Dye Terminators, Perkin Elmer Co.). Sequencing reactions were analyzed on an ABI377 DNA sequencer (Perkin Elmer Co.).

DNA sequences adjacent to the site of the insertion were collected and used to search DNA and protein databases using the BLAST algorithms (Altschul et al., supra). A single insertion of SIF into the Magnaporthe grisea TPS1 gene was chosen for further analysis. This construct was designated cpgmr0012027a12 and it contains the SIF transposon approximately between amino acids 210 and 211 relative to the Aspergillus niger homologue, TPSA (total length: 517 amino acids, GENBANK: 2499017).

### EXAMPLE 5

### Preparation of TPS1 Cosmid DNA and Transformation of Magnaporthe grisea

Cosmid DNA from the TPS1 transposon tagged cosmid clone was prepared using QIAGEN Plasmid Maxi Kit (Qiagen), and digested by PI-PspI (New England Biolabs, Inc.). Fungal electro-transformation was performed essentially as described (Wu et al., 10 MPMI 700 (1997)). Briefly, M. grisea strain Guy 11 was grown in complete liquid media (Talbot et al., 5 Plant Cell 1575 (1993)) shaking at 120 rpm for 3 days at 25° C. in the dark. Mycelia was harvested and washed with sterile H₂O and digested with 4 mg/ml beta-glucanase (InterSpex) for 4-6 hours to generate protoplasts. Protoplasts were collected by centrifugation and resuspended in 20% sucrose at a concentration of 2×10⁸ protoplasts/ml. 50 ul of protoplast suspension was mixed with 10-20 ug of the cosmid DNA and pulsed using a Gene Pulser II instrument (BioRad) set with the following parameters: 200 ohm, 25 uF, and 0.6 kV. Transformed protoplasts were regenerated in complete agar media (Talbot et al., supra) with the addition of 20% sucrose for one day, then overlayed with CM agar media containing hygromycin B (250 ug/ml) to select transformants. Transformants were screened for homologous recombination events in the target gene by PCR (Hamer et al., supra). Two independent strains were identified and are hereby referred to as K1-28 and K1-30, respectively.

### EXAMPLE 6

### Effect of Transposon Insertion on Magnaporthe Pathogenicity

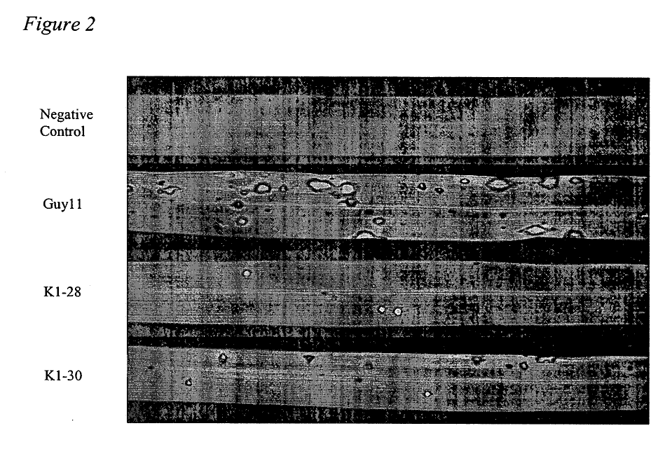

The target fungal strains, K1-28 and K1-30, obtained in Example 5 and the wild-type strain, Guy11, were subjected to a pathogenicity assay to observe infection over a 1-week period. Rice infection assays were performed using Indian rice cultivar CO39 essentially as described in Valent et al. (Valent et al., 127 Genetics 87 (1991)). All three strains were grown for spore production on complete agar media. Spores were harvested and the concentration of spores adjusted for whole plant inoculations. Two-week-old seedlings of cultivar CO39 were sprayed with 12 ml of conidial suspension (5×10⁴ conidia per ml in 0.01% Tween-20 solution). The inoculated plants were incubated in a dew chamber at 27° C. in the dark for 36 hours, and transferred to a growth chamber (27° C. 12 hours/21° C. 12 hours at 70% humidity) for an additional 5.5 days. Leaf samples were taken at 3, 5, and 7 days post-inoculation and examined for signs of successful infection (i.e. lesions). FIG. 2 shows the effects of TPS1 gene disruption on Magnaporthe infection at five days post-inoculation.

### EXAMPLE 7

### Cloning and Expression Strategies, Extraction and Purification of Trehalose-6-Phosphate Synthase Protein

The following protocol may be employed to obtain a purified Trehalose-6-Phosphate Synthase protein.

Cloning and Expression Strategies:

A TPS1 cDNA gene can be cloned into E. coli (pET vectors-Novagen), Baculovirus (Pharmingen) and Yeast (Invitrogen) expression vectors containing His/fusion protein tags, and the expression of recombinant protein can be evaluated by SDS-PAGE and Western blot analysis.

Extraction:

Extract recombinant protein from 250 ml cell pellet in 3 ml of extraction buffer by sonicating 6 times, with 6 second pulses at 4° C. Centrifuge extract at 15000×g for 10 minutes and collect supernatant. Assess biological activity of the recombinant protein by activity assay.

Purification:

Purify recombinant protein by Ni-NTA affinity chromatography (Qiagen).

Purification protocol (perform all steps at 4° C.):

Use 3 ml Ni-beads

Equilibrate column with the buffer

Load protein extract

Wash with the equilibration buffer

Elute bound protein with 0.5 M imidazole

### EXAMPLE 8

### Assays for Testing Binding of Test Compounds to Trehalose-6-Phosphate Synthase

The following protocol may be employed to identify test compounds that bind to the Trehalose-6-Phosphate Synthase protein.

Purified full-length Trehalose-6-Phosphate Synthase polypeptide with a His/fusion protein tag (Example 8) is bound to a HISGRAB Nickel Coated Plate (Pierce, Rockford, Ill.) following manufacturer's instructions.

Buffer conditions are optimized (e.g. ionic strength or pH, Wolschek and Kubicek (1997) J Biol Chem 272: 2729-35 (PMID: 9006911)) for binding of radiolabeled ¹⁴C-labeled D-glucose-6-phosphate (Moravek Biochemicals) to the bound Trehalose-6-Phosphate Synthase.

Screening of test compounds is performed by adding test compound and ¹⁴C-labeled D-glucose-6-phosphate (Moravek Biochemicals) to the wells of the HISGRAB plate containing bound Trehalose-6-Phosphate Synthase.

The wells are washed to remove excess labeled ligand and scintillation fluid (SCINTIVERSE, Fisher Scientific) is added to each well.

The plates are read in a microplate scintillation counter.

Candidate compounds are identified as wells with lower radioactivity as compared to control wells with no test compound added.

Additionally, a purified polypeptide comprising 10-50 amino acids from the M. grisea Trehalose-6-Phosphate Synthase is screened in the same way. A polypeptide comprising 10-50 amino acids is generated by subcloning a portion of the TPS1 gene into a protein expression vector that adds a His-Tag when expressed (see Example 7). Oligonucleotide primers are designed to amplify a portion of the TPS1 gene using the polymerase chain reaction amplification method. The DNA fragment encoding a polypeptide of 10-50 amino acids is cloned into an expression vector, expressed in a host organism and purified as described in Example 8 above.

Test compounds that bind TPS1 are further tested for antibiotic activity. M grisea is grown as described for spore production on oatmeal agar media (Talbot et al., supra). Spores are harvested into minimal media to a concentration of 2×10⁵ spores/ml and the culture is divided. Id. The test compound is added to one culture to a final concentration of 20-100 μg/ml. Solvent only is added to the second culture. The plates are incubated at 25° C. for seven days and optical density measurements at 590 nm are taken daily. The growth curves of the solvent control sample and the test compound sample are compared. A test compound is an antibiotic candidate if the growth of the culture containing the test compound is less than the growth of the control culture.

Test compounds that bind TPS1 are further tested for antipathogenic activity. M grisea is grown as described for spore production on oatmeal agar media (Talbot et al., supra). Spores are harvested into water with 0.01% Tween 20 to a concentration of 5×10⁴ spores/ml and the culture is divided. Id. The test compound is added to one culture to a final concentration of 20-100 μg/ml. Solvent only is added to the second culture. Rice infection assays are performed using Indian rice cultivar CO39 essentially as described in Valent et al., supra). Two-week-old seedlings of cultivar CO39 are sprayed with 12 ml of conidial suspension. The inoculated plants are incubated in a dew chamber at 27° C. in the dark for 36 hours, and transferred to a growth chamber (27° C. 12 hours/21° C. 12 hours at 70% humidity) for an additional 5.5 days. Leaf samples are examined at 5 days post-inoculation to determine the extent of pathogenicity as compared to the control samples.

Alternatively, antipathogenic activity can be assessed using an excised leaf pathogenicity assay. Spore suspensions are prepares in water only to a concentration of 5×10⁴ spores/ml and the culture is divided. The test compound is added to one culture to a final concentration of 20-100 μg/ml. Solvent only is added to the second culture. Detached leaf assays are performed by excising 1 cm segments of rice leaves from Indian rice cultivar CO39 and placing them on 1% agarose in water. 10 μl of each spore suspension is place on the leaf segments and the samples are incubated at 25° C. for 5 days in the dark. Leaf samples are examined at 5 days post-inoculation to determine the extent of pathogenicity as compared to the control samples.

### EXAMPLE 9

### Assays for Testing Inhibitors or Candidates for Inhibition of Trehalose-6-Phosphate Synthase Activity

The enzymatic activity of Trehalose-6-Phosphate Synthase is determined in the presence and absence of candidate compounds in a suitable reaction mixture, such as described by Wolschek and Kubicek. Wolschek & Kubicek, 272 J. Biol. Chem. 2729-35 (1997). Candidate compounds are identified when a decrease in products or a lack of decrease in substrates is detected with the reaction proceeding in either direction.

Additionally, the enzymatic activity of a polypeptide comprising 10-50 amino acids from the M. grisea Trehalose-6-Phosphate Synthase is determined in the presence and absence of candidate compounds in a suitable reaction mixture, such as described by Wolschek and Kubicek. Id. A polypeptide comprising 10-50 amino acids is generated by subcloning a portion of the TPS1 gene into a protein expression vector that adds a His-Tag when expressed (see Example 8). Oligonucleotide primers are designed to amplify a portion of the TPS1 gene using polymerase chain reaction amplification method. The DNA fragment encoding a polypeptide of 10-50 amino acids is cloned into an expression vector, expressed and purified as described in Example 8 above.

Test compounds identified as inhibitors of TPS1 activity are further tested for antibiotic activity. Magnaporthe grisea fungal cells are grown under standard fungal growth conditions that are well known and described in the art. M. grisea is grown as described for spore production on oatmeal agar media (Talbot et al., supra). Spores are harvested into minimal media to a concentration of 2×10⁵ spores/ml and the culture is divided. Id. The test compound is added to one culture to a final concentration of 20-100 μg/ml. Solvent only is added to the second culture. The plates are incubated at 25° C. for seven days and optical density measurements at 590 nm are taken daily. The growth curves of the solvent control sample and the test compound sample are compared. A test compound is an antibiotic candidate if the growth of the culture containing the test compound is less than the growth of the control culture.

Test compounds identified as inhibitors of TPS1 activity are further tested for antipathogenic activity. M. grisea is grown as described for spore production on oatmeal agar media (Talbot et al., supra). Spores are harvested into water with 0.01% Tween 20 to a concentration of 5×10⁴ spores/ml and the culture is divided. Id. The test compound is added to one culture to a final concentration of 20-100 μg/ml. Solvent only is added to the second culture. Rice infection assays are performed using Indian rice cultivar CO39 essentially as described in Valent et al., supra. Two-week-old seedlings of cultivar CO39 are sprayed with 12 ml of conidial suspension. The inoculated plants are incubated in a dew chamber at 27° C. in the dark for 36 hours, and transferred to a growth chamber (27° C. 12 hours/21° C. 12 hours at 70% humidity) for an additional 5.5 days. Leaf samples are examined at 5 days post-inoculation to determine the extent of pathogenicity as compared to the control samples.

Alternatively, antipathogenic activity can be assessed using an excised leaf pathogenicity assay. Spore suspensions are prepared in water only to a concentration of 5×10⁴ spores/ml and the culture is divided. The test compound is added to one culture to a final concentration of 20-100 μg/ml. Solvent only is added to the second culture. Detached leaf assays are performed by excising 1 cm segments of rice leaves from Indian rice cultivar CO39 and placing them on 1% agarose in water. 10 μl of each spore suspension is place on the leaf segments and the samples are incubated at 25° C. for 5 days in the dark. Leaf samples are examined at 5 days post-inoculation to determine the extent of pathogenicity as compared to the control samples.

### EXAMPLE 10

### Assays for Testing Compounds for Alteration of Trehalose-6-Phosphate Synthase Gene Expression

Magnaporthe grisea fungal cells are grown under standard fungal growth conditions that are well known and described in the art. Wild-type M. grisea spores are harvested from cultures grown on complete agar or oatmeal agar media after growth for 10-13 days in the light at 25° C. using a moistened cotton swab. The concentration of spores is determined using a hemacytometer and spore suspensions are prepared in a minimal growth medium to a concentration of 2×10⁵ spores per ml. 25 ml cultures are prepared to which test compounds will be added at various concentrations. A culture with no test compound present is included as a control. The cultures are incubated at 25° C. for 3 days after which test compound or solvent only control is added. The cultures are incubated an additional 18 hours. Fungal mycelia is harvested by filtration through Miracloth (CalBiochem, La Jolla, Calif.), washed with water, and frozen in liquid nitrogen. Total RNA is extracted with TRIZOL Reagent using the methods provided by the manufacturer (Life Technologies, Rockville, Md.). Expression is analyzed by Northern analysis of the RNA samples as described (Sambrook et al., supra) using a radiolabeled fragment of the TPS1 gene as a probe. Test compounds resulting in a reduced level of TPS1 mRNA relative to the untreated control sample are identified as candidate antibiotic compounds.

Test compounds identified as inhibitors of TPS1 expression are further tested for antibiotic activity. Magnaporthe grisea fungal cells are grown under standard fungal growth conditions that are well known and described in the art. M. grisea is grown as described for spore production on oatmeal agar media (Talbot et al., supra). Spores are harvested into minimal media to a concentration of 2×10⁵ spores/ml and the culture is divided. Id. The test compound is added to one culture to a final concentration of 20-100 μg/ml. Solvent only is added to the second culture. The plates are incubated at 25° C. for seven days and optical density measurements at 590 nm are taken daily. The growth curves of the solvent control sample and the test compound sample are compared. A test compound is an antibiotic candidate if the growth of the culture containing the test compound is less than the growth of the control culture.

Test compounds identified as inhibitors of TPS1 gene expression are further tested for antipathogenic activity. M. grisea is grown as described for spore production on oatmeal agar media (Talbot et al., supra). Spores are harvested into water with 0.01% Tween 20 to a concentration of 5×10⁴ spores/ml and the culture is divided. Id. The test compound is added to one culture to a final concentration of 20-100 μg/ml. Solvent only is added to the second culture. Rice infection assays are performed using Indian rice cultivar CO39 essentially as described in Valent et al., supra. Two-week-old seedlings of cultivar CO39 are sprayed with 12 ml of conidial suspension. The inoculated plants are incubated in a dew chamber at 27° C. in the dark for 36 hours, and transferred to a growth chamber (27° C. 12 hours/21° C. 12 hours at 70% humidity) for an additional 5.5 days. Leaf samples are examined at 5 days post-inoculation to determine the extent of pathogenicity as compared to the control samples.

Alternatively, antipathogenic activity can be assessed using an excised leaf pathogenicity assay. Spore suspensions are prepared in water only to a concentration of 5×10⁴ spores/ml and the culture is divided. The test compound is added to one culture to a final concentration of 20-100 μg/ml. Solvent only is added to the second culture. Detached leaf assays are performed by excising 1 cm segments of rice leaves from Indian rice cultivar CO39 and placing them on 1% agarose in water. 10 μl of each spore suspension is place on the leaf segments and the samples are incubated at 25° C. for 5 days in the dark. Leaf samples are examined at 5 days post-inoculation to determine the extent of pathogenicity as compared to the control samples.

### EXAMPLE 11

### In Vivo Cell Based Assay Screening Protocol with a Fungal Strain Containing a Mutant form of Trehalose-6-Phosphate Synthase with No Activity or Reduced Activity

Magnaporthe grisea fungal cells containing a mutant form of the TPS1 gene which abolishes enzyme activity, such as a gene containing a transposon insertion (see Examples 4 and 5), are grown under standard fungal growth conditions that are well known and described in the art. Magnaporthe grisea spores are harvested from cultures grown on complete agar medium after growth for 10-13 days in the light at 25° C. using a moistened cotton swab. The concentration of spores is determined using a hemacytometer and spore suspensions are prepared in a minimal growth medium to a concentration of 2×10⁵ spores per ml. Approximately 4×10⁴ spores are added to each well of 96-well plates to which a test compound is added (at varying concentrations). The total volume in each well is 200 μl. Wells with no test compound present (growth control), and wells without cells are included as controls (negative control). The plates are incubated at 25° C. for seven days and optical density measurements at 590 nm are taken daily. Wild-type cells are screened under the same conditions. The effect of each compound on the mutant and wild-type fungal strains is measured against the growth control and the percent of inhibition is calculated as the OD₅₉₀ (fungal strain plus test compound)/OD₅₉₀ (growth control)×100. The percent of growth inhibition as a result of a test compound on a fungal strain and that on the wild-type cells are compared. Compounds that show differential growth inhibition between the mutant and the wild-type are identified as potential antifingal compounds. Similar protocols may be found in Kirsch & DiDomenico, 26 Biotechnology 177 (1994).

Test compounds that produce a differential growth response between the mutant and wild-type fungal strain are further tested for antipathogenic activity. Each M. grisea strain is grown as described for spore production on oatmeal agar media (Talbot et al., supra). Spores for each strain are harvested into water with 0.01% Tween 20 to a concentration of 5×10⁴ spores/ml and the culture is divided. Id. The test compound is added to one culture to a final concentration of 20-100 μg/ml. Solvent only is added to the second culture. Rice infection assays are performed using Indian rice cultivar CO39 essentially as described in Valent et al., supra. Two-week-old seedlings of cultivar CO39 are sprayed with 12 ml of conidial suspension. The inoculated plants are incubated in a dew chamber at 27° C. in the dark for 36 hours, and transferred to a growth chamber (27° C. 12 hours/21° C. 12 hours 70% humidity) for an additional 5.5 days. Leaf samples are examined at 5 days post-inoculation to determine the extent of pathogenicity of the mutant and wild-type fungal strains as compared to their untreated control samples.

Alternatively, antipathogenic activity can be assessed using an excised leaf pathogenicity assay. Spore suspensions are prepared in water only to a concentration of 5×10⁴ spores/ml and the culture is divided. The test compound is added to one culture to a final concentration of 20-100 μg/ml. Solvent only is added to the second culture. Detached leaf assays are performed by excising 1 cm segments of rice leaves from Indian rice cultivar CO39 and placing them on 1% agarose in water. 10 μl of each spore suspension is place on the leaf segments and the samples are incubated at 25° C. for 5 days in the dark. Leaf samples are examined at 5 days post-inoculation to determine the extent of pathogenicity as compared to the untreated control samples.

### EXAMPLE 12

### In Vivo Cell Based Assay Screening Protocol with a Fungal Strain Containing a Mutant form of a Trehalose Biosynthetic Gene with No Activity or Reduced Activity

Magnaporthe grisea fungal cells containing a mutant form of a gene in the trehalose biosynthetic pathway (e.g. Trehalose-Phosphatase (E.C. 3.1.3.12)) are grown under standard fungal growth conditions that are well known and described in the art. Magnaporthe grisea spores are harvested from cultures grown on complete agar medium containing after growth for 10-13 days in the light at 25° C. using a moistened cotton swab. The concentration of spores is determined using a hemacytometer and spore suspensions are prepared in a minimal growth medium to a concentration of 2×10⁵ spores per ml. Approximately 4×10⁴ spores or cells are harvested and added to each well of 96-well plates to which growth media is added in addition to an amount of test compound (at varying concentrations). The total volume in each well is 200 μl. Wells with no test compound present, and wells without cells are included as controls. The plates are incubated at 25° C. for seven days and optical density measurements at 590 nm are taken daily. Wild-type cells are screened under the same conditions. The effect of each compound on the mutant and wild-type fungal strains is measured against the growth control and the percent of inhibition is calculated as the OD₅₉₀ (fungal strain plus test compound)/OD₅₉₀ (growth control)×100. The percent of growth inhibition as a result of a test compound on a fungal strain and that on the wild-type cells are compared. Compounds that show differential growth inhibition between the mutant and the wild-type are identified as potential antifungal compounds. Similar protocols may be found in Kirsch & DiDomenico, supra.

Test compounds that produce a differential growth response between the mutant and wild-type fungal strain are further tested for antipathogenic activity. Each M. grisea strain is grown as described for spore production on oatmeal agar media (Talbot et al., supra). Spores for each strain are harvested into water with 0.01% Tween 20 to a concentration of 5×10⁴ spores/ml and the culture is divided. Id. The test compound is added to one culture to a final concentration of 20-100 μg/ml. Solvent only is added to the second culture. Rice infection assays are performed using Indian rice cultivar CO39 essentially as described in Valent et al., supra. Two-week-old seedlings of cultivar CO39 are sprayed with 12 ml of conidial suspension. The inoculated plants are incubated in a dew chamber at 27° C. in the dark for 36 hours, and transferred to a growth chamber (27° C. 12 hours/21° C. 12 hours at 70% humidity) for an additional 5.5 days. Leaf samples are examined at 5 days post-inoculation to determine the extent of pathogenicity of the mutant and wild-type fungal strains as compared to their untreated control samples.

Alternatively, antipathogenic activity can be assessed using an excised leaf pathogenicity assay. Spore suspensions are prepared in water only to a concentration of 5×10⁴ spores/ml and the culture is divided. The test compound is added to one culture to a final concentration of 20-100 μg/ml. Solvent only is added to the second culture. Detached leaf assays are performed by excising 1 cm segments of rice leaves from Indian rice cultivar CO39 and placing them on 1% agarose in water. 10 μl of each spore suspension is place on the leaf segments and the samples are incubated at 25° C. for 5 days in the dark. Leaf samples are examined at 5 days post-inoculation to determine the extent of pathogenicity as compared to the untreated control samples.

### EXAMPLE 13

### In Vivo Cell Based Assay Screening Protocol with a Fungal Strain Containing a Fungal TPS1 and a Second Fungal Strain Containing a Heterologous TPS1 Gene

Wild-type Magnaporthe grisea fungal cells and M. grisea fungal cells lacking a functional TPS1 gene and containing a heterologous TPS1 gene from Aspergillus niger (Genbank 2499017, 76.2% protein sequence identity) are grown under standard fungal growth conditions that are well known and described in the art. A M. grisea strain carrying a heterologous TPS1 gene is made as follows:

A M. grisea strain is made with a nonfunctional TPS1 gene, such as one containing a transposon insertion in the native gene (see Examples 4 and 5).

A construct containing a heterologous TPS1 gene is made by cloning the TPSA gene from Aspergillus niger into a fungal expression vector containing a trpC promoter and terminator (e.g. Carroll et al., 41 Fungal Gen. News Lett. 22 (1994) (describing pCB1003) using standard molecular biology techniques that are well known and described in the art (Sambrook et al., supra).

The said construct is used to transform the M. grisea strain lacking a functional TPS1 gene (see Example 5). Transformants are selected on minimal agar medium containing a selectable marker. Only transformants carrying a TPS1 gene construct with the selectable marker will grow.

Wild-type strains of Magnaporthe grisea and strains containing a heterologous form of TPS1 are grown under standard fungal growth conditions that are well known and described in the art. Magnaporthe grisea spores are harvested from cultures grown on complete agar medium after growth for 10-13 days in the light at 25° C. using a moistened cotton swab. The concentration of spores is determined using a hemacytometer and spore suspensions are prepared in a minimal growth medium to a concentration of 2×10⁵ spores per ml. Approximately 4×10⁴ spores or cells are harvested and added to each well of 96-well plates to which growth media is added in addition to an amount of test compound (at varying concentrations). The total volume in each well is 200 μl. Wells with no test compound present, and wells without cells are included as controls. The plates are incubated at 25° C. for seven days and optical density measurements at 590 nm are taken daily. The effect of each compound on the wild-type and heterologous fungal strains is measured against the growth control and the percent of inhibition is calculated as the OD₅₉₀ (fungal strain plus test compound)/OD₅90 (growth control)×100. The percent of growth inhibition as a result of a test compound on the wild-type and heterologous fungal strains are compared. Compounds that show differential growth inhibition between the wild-type and heterologous strains are identified as potential antifungal compounds with specificity to the native or heterologous TPS1 gene products. Similar protocols may be found in Kirsch & DiDomenico, supra.

Test compounds that produce a differential growth response between the strain containing a heterologous gene and strain containing a fungal gene are further tested for antipathogenic activity. Each M. grisea strain is grown as described for spore production on oatmeal agar media (Talbot et al., supra). Spores for each strain are harvested into water with 0.01% Tween 20 to a concentration of 5×10⁴ spores/ml and the culture is divided. Id. The test compound is added to one culture to a final concentration of 20-100 μg/ml. Solvent only is added to the second culture. Rice infection assays are performed using Indian rice cultivar CO39 essentially as described in Valent et al., supra. Two-week-old seedlings of cultivar CO39 are sprayed with 12 ml of conidial suspension. The inoculated plants are incubated in a dew chamber at 27° C. in the dark for 36 hours, and transferred to a growth chamber (27° C. 12 hours/21° C. 12 hours at 70% humidity) for an additional 5.5 days. Leaf samples are examined at 5 days post-inoculation to determine the extent of pathogenicity of the mutant and wild-type fungal strains as compared to their untreated control samples.

Alternatively, antipathogenic activity can be assessed using an excised leaf pathogenicity assay. Spore suspensions are prepared in water only to a concentration of 5×10⁴ spores/ml and the culture is divided. The test compound is added to one culture to a final concentration of 20-100 μg/ml. Solvent only is added to the second culture. Detached leaf assays are performed by excising 1 cm segments of rice leaves from Indian rice cultivar CO39 and placing them on 1% agarose in water. 10 μl of each spore suspension is place on the leaf segments and the samples are incubated at 25° C. for 5 days in the dark. Leaf samples are examined at 5 days post-inoculation to determine the extent of pathogenicity as compared to the untreated control samples.

Published references and patent publications cited herein are incorporated by reference as if terms incorporting the same were provided upon each occurrence of the individual reference or patent document. While the foregoing describes certain embodiments of the invention, it will be understood by those skilled in the art that variations and modifications may be made that will fall within the scope of the invention. The foregoing examples are intended to exemplify various specific embodiments of the invention and do not limit its scope in any manner.

What is claimed is:

1. A method for identifying a test compound as a candidate for an antibiotic, comprising:
a) contacting a Trehalose-6-Phosphate Synthase polypeptide with a test compound; and
b) detecting the presence or absence of binding between the test compound and the Trehalose-6-Phosphate Synthase polypeptide, wherein binding indicates that the test compound is a candidate for an antibiotic.

2. The method of claim 1, wherein said Trehalose-6-Phosphate Synthase polypeptide is a fungal Trehalose-6-Phosphate Synthase polypeptide.

3. The method of claim 1, wherein said Trehalose-6-Phosphate Synthase polypeptide is a Magnaporthe Trehalose-6-Phosphate Synthase polypeptide.

4. The method of claim 1, wherein said Trehalose-6-Phosphate Synthase polypeptide is SEQ ID NO: 3.

5. A method for determining whether the antibiotic candidate of claim 1 has antifungal activity, further comprising: contacting a fungus or fungal cells with the antibiotic candidate and detecting the decrease in growth, viability, or pathogenicity of the fungus or fungal cells.

6. A method for identifying a test compound as a candidate for an antibiotic, comprising:
a) contacting a test compound with at least one polypeptide selected from the group consisting of: a polypeptide having at least ten consecutive amino acids of a fungal Trehalose-6-Phosphate Synthase, a polypeptide having at least 50% sequence identity with a fungal Trehalose-6-Phosphate Synthase, and a polypeptide having at least 10% of the activity thereof; and
b) detecting the presence and/or absence of binding between the test compound and the polypeptide, wherein binding indicates that the test compound is a candidate for an antibiotic.

7. A method for determining whether the antibiotic candidate of claim 6 has antifungal activity, further comprising: contacting a fungus or fungal cells with the antibiotic candidate and detecting a decrease in growth, viability, or pathogenicity of the fungus or fungal cells.

8. A method for identifying a test compound as a candidate for an antibiotic, comprising:
a) contacting UDP-glucose and D-glucose-6-phosphate with a Trehalose-6-Phosphate Synthase;
b) contacting UDP-glucose and D-glucose-6-phosphate with Trehalose-6-Phosphate Synthase and a test compound; and
c) determining the change in concentration for at least one of the following: UDP, UDP-glucose, D-glucose-6-phosphate, and/or alpha, alpha′-trehalose-6-phosphate, wherein a change in concentration for any of the above substances between steps (a) and (b) indicates that the test compound is a candidate for an antibiotic.

9. The method of claim 8, wherein the Trehalose-6-Phosphate Synthase is a fungal Trehalose-6-Phosphate Synthase.

10. The method of claim 8, wherein the Trehalose-6-Phosphate Synthase is a Magnaporthe Trehalose-6-Phosphate Synthase.

11. The method of claim 8, wherein the Trehalose-6-Phosphate Synthase is SEQ ID NO: 3.

12. A method for determining whether the antibiotic candidate of claim 8 has antifungal activity, further comprising: contacting a fungus or fungal cells with the antibiotic candidate and detecting a decrease in growth, viability, or pathogenicity of the fungus or fungal cells.

13. A method for identifying a test compound as a candidate for an antibiotic, comprising:
a) contacting UDP and alpha, alpha′-trehalose-6-phosphate with a Trehalose-6-Phosphate Synthase;
b) contacting UDP and alpha, alpha′-trehalose-6-phosphate with a Trehalose-6-Phosphate Synthase and a test compound; and
c) determining the change in concentration for at least one of the following: UDP, UDP-glucose, D-glucose-6-phosphate, and/or alpha, alpha′-trehalose-6-phosphate, wherein a change in concentration for any of the above substances between steps (a) and (b) indicates that the test compound is a candidate for an antibiotic.

14. The method of claim 13, wherein the Trehalose-6-Phosphate Synthase is a fungal Trehalose-6-Phosphate Synthase.

15. The method of claim 13, wherein the Trehalose-6-Phosphate Synthase is a Magnaporthe Trehalose-6-Phosphate Synthase.

16. The method of claim 13, wherein the Trehalose-6-Phosphate Synthase is SEQ ID NO: 3.

17. A method for determining whether the antibiotic candidate of claim 13 has antifungal activity, further comprising: contacting a fungus or fungal cells with the antibiotic candidate and detecting a decrease in growth, viability, or pathogenicity of the fungus or fungal cells.

18. A method for identifying a test compound as a candidate for an antibiotic, comprising:
a) contacting UDP-glucose and D-glucose-6-phosphate with a polypeptide selected from the group consisting of: a polypeptide having at least 50% sequence identity with Trehalose-6-Phosphate Synthase, a polypeptide having at least 50% sequence identity with a Trehalose-6-Phosphate Synthase and having at least 10% of the activity thereof, and a polypeptide comprising at least 100 consecutive amino acids of a Trehalose-6-Phosphate Synthase;
b) contacting UDP-glucose and D-glucose-6-phosphate with said polypeptide and a test compound; and
c) determining the change in concentration for at least one of the following: UDP, UDP-glucose, D-glucose-6-phosphate, and/or alpha, alpha′-trehalose-6-phosphate, wherein a change in concentration for any of the above substances between steps (a) and (b) indicates that the test compound is a candidate for an antibiotic.

19. A method for identifying a test compound as a candidate for an antibiotic, comprising:
a) contacting UDP and alpha, alpha′-trehalose-6-phosphate with a polypeptide selected from the group consisting of: a polypeptide having at least 50% sequence identity with a Trehalose-6-Phosphate Synthase, a polypeptide having at least 50% sequence identity with a Trehalose-6-Phosphate Synthase and at least 10% of the activity thereof, and a polypeptide comprising at least 100 consecutive amino acids of a Trehalose-6-Phosphate Synthase;
b) contacting UDP and alpha, alpha′-trehalose-6-phosphate, with said polypeptide and a test compound; and
c) determining the change in concentration for at least one of the following: UDP, UDP-glucose, D-glucose-6-phosphate, and/or alpha, alpha′-trehalose-6-phosphate, wherein a change in concentration for any of the above substances between steps (a) and (b) indicates that the test compound is a candidate for an antibiotic.

20. A method for identifying a test compound as a candidate for an antibiotic, comprising:
a) measuring the expression of a Trehalose-6-Phosphate Synthase in a cell, cells, tissue, or an organism in the absence of a test compound;
b) contacting the cell, cells, tissue, or organism with the test compound and measuring the expression of the Trehalose-6-Phosphate Synthase in the cell, cells, tissue, or organism; and
c) comparing the expression of Trehalose-6-Phosphate Synthase in steps (a) and (b), wherein a lower expression in the presence of the test compound indicates that the test compound is a candidate for an antibiotic.

21. The method of claim 20, wherein the cell, cells, tissue, or organism is, or is derived from a fungus.

22. The method of claim 20, wherein the cell, cells, tissue, or organism is, or is derived from a Magnaporthe fungus or fungal cell.

23. The method of claim 20, wherein the Trehalose-6-Phosphate Synthase is SEQ ID NO: 3.

24. The method of claim 20, wherein the expression of Trehalose-6-Phosphate Synthase is measured by detecting TPS1 mRNA.

25. The method of claim 20, wherein the expression of Trehalose-6-Phosphate Synthase is measured by detecting Trehalose-6-Phosphate Synthase polypeptide.

26. A method for identifying a test compound as a candidate for an antibiotic, comprising:
a) providing cells having one form of a Trehalose-6-Phosphate Synthase gene, and providing comparison cells having a different form of a Trehalose-6-Phosphate Synthase gene; and
b) contacting the cells and the comparison cells with a test compound and determining the growth of the cells and comparison cells in the presence of the test compound, wherein a difference in growth between the cells and the comparison cells in the presence of the compound indicates that the compound is a candidate for an antibiotic.

27. The method of claim 26, wherein the cells and the comparison cells are fungal cells.

28. The method of claim 26, wherein the cells and the comparison cells are Magnaporthe cells.

29. The method of claim 26, wherein the form and the different form of the Trehalose-6-Phosphate Synthase are fungal Trehalose-6-Phosphate Synthases.

30. The method of claim 26, wherein at least one of the protein forms is a Magnaporthe Trehalose-6-Phosphate Synthase.

31. The method of claim 26, wherein the form and the different form of the Trehalose-6-Phosphate Synthase are non-fungal Trehalose-6-Phosphate Synthases.

32. The method of claim 26, wherein one form of the Trehalose-6-Phosphate Synthase is a fungal Trehalose-6-Phosphate Synthase, and the different form is a non-fungal Trehalose-6-Phosphate Synthase.

33. A method for identifying a test compound as a candidate for an antibiotic, comprising:
a) providing cells having one form of a Trehalose-6-Phosphate Synthase gene, and providing comparison cells having a different form of a Trehalose-6-Phosphate Synthase gene; and
b) contacting the cells and the comparison cells with a test compound and determining the pathogenicity of the cells and comparison cells in the presence of the test compound, wherein a difference in pathogenicity between the cells and the comparison cells in the presence of the compound indicates that the compound is a candidate for an antibiotic.

34. The method of claim 33, wherein the cells and the comparison cells are fungal cells.

35. The method of claim 33, wherein the cells and the comparison cells are Magnaporthe cells.

36. The method of claim 33, wherein the form and the different form of the Trehalose-6-Phosphate Synthase are fungal Trehalose-6-Phosphate Synthases.

37. The method of claim 33, wherein at least one of the protein forms is a Magnaporthe Trehalose-6-Phosphate Synthase.

38. The method of claim 33, wherein the form and the different form of the Trehalose-6-Phosphate Synthase are non-fungal Trehalose-6-Phosphate Synthases.

39. The method of claim 33, wherein one form of the Trehalose-6-Phosphate Synthase is a fungal Trehalose-6-Phosphate Synthase, and the different form is a non-fungal Trehalose-6-Phosphate Synthase.

40. A method for identifying a test compound as a candidate for an antibiotic, comprising:
a) providing cells having one form of a gene in the trehalose biochemical and/or genetic pathway and providing comparison cells having a different form of the gene;
b) contacting the cells and the comparison cells with a test compound; and
c) determining the growth of the cells and the comparison cells in the presence of the test compound, wherein a difference in growth between the cells and the comparison cells in the presence of the test compound indicates that the test compound is a candidate for an antibiotic.

41. The method of claim 40, wherein the cells and the comparison cells are fungal cells.

42. The method of claim 40, wherein the cells and the comparison cells are Magnaporthe cells.

43. The method of claim 40, wherein the form and the different form of the trehalose biosynthesis gene are fungal trehalose biosynthesis genes.

44. The method of claim 40, wherein at least one of the protein forms is a Magnaporthe trehalose biosynthesis gene.

45. The method of claim 40, wherein the form and the different form of the trehalose biosynthesis genes are non-fungal trehalose biosynthesis genes.

46. The method of claim 40, wherein one form of the trehalose biosynthesis gene is a fungal trehalose biosynthesis gene, and the different form is a non-fungal trehalose biosynthesis gene.

47. A method for determining whether the antibiotic candidate of claim 40 has antifungal activity, further comprising: contacting a fungus or fungal cells with the antibiotic candidate and detecting a decrease in growth, viability, or pathogenicity of the fungus or fungal cells, wherein a decrease in growth, viability, or pathogenicity of the fungus or fungal cells indicates that the antibiotic candidate has antifungal activity.

48. A method for identifying a test compound as a candidate for an antibiotic, comprising:
a) providing cells having one form of a gene in the trehalose biochemical and/or genetic pathway and providing comparison cells having a different form of the gene;
b) contacting the cells and the comparison cells with a test compound; and
c) determining the pathogenicity of the cells and the comparison cells in the presence of the test compound, wherein a difference in pathogenicity between the cells and the comparison cells in the presence of the test compound indicates that the test compound is a candidate for an antibiotic.

49. The method of claim 48, wherein the cells and the comparison cells are fungal cells.

50. The method of claim 48, wherein the cells and the comparison cells are Magnaporthe cells.

51. The method of claim 48, wherein the form and the different form of the trehalose biosynthesis gene are fungal trehalose biosynthesis genes.

52. The method of claim 48, wherein at least one of the protein forms is a Magnaporthe trehalose biosynthesis gene.

53. The method of claim 48, wherein the form and the different form of the trehalose biosynthesis genes are non-fungal trehalose biosynthesis genes.

54. The method of claim 48, wherein one form of the trehalose biosynthesis gene is a fungal trehalose biosynthesis gene, and the different form is a non-fungal trehalose biosynthesis gene.

55. A method for determining whether the antibiotic candidate of claim 48 has antifungal activity, further comprising: contacting a fungus or fungal cells with the antibiotic candidate and detecting a decrease in growth, viability, or pathogenicity of the fungus or fungal cells, wherein a decrease in growth, viability, or pathogenicity of the fungus or fungal cells indicates that the antibiotic candidate has antifungal activity.

56. An isolated nucleic acid comprising a nucleotide sequence that encodes a polypeptide of SEQ ID NO: 3.

57. The nucleic acid of claim 56, comprising the nucleotide sequence of SEQ ID NO: 1.

58. An expression cassette comprising the nucleic acid of claim 57.

59. The isolated nucleic acid of claim 56, comprising a nucleotide sequence with at least 50% to at least 95% sequence identity to SEQ ID NO: 1.

60. A polypeptide consisting essentially of the amino acid sequence of SEQ ID NO: 3.

61. A polypeptide comprising the amino acid sequence of SEQ ID NO: 3.
